(19) United States
(12) Patent Application Publication
(10) Pub. No.: US 2009/0171341 A1
(43) Pub. Date: Jul. 2, 2009

(54) DISPERSIVE RETURN ELECTRODE AND METHODS

(76) Inventors: Karl Pope, San Mateo, CA (US); Amogh Kothare, Redwood City, CA (US); Dean A. MacFarland, Magnolia, MA (US)

Correspondence Address: Cutera c/o MoFo, 425 Market Street, San Francisco, CA, 94105-2482, US

(21) Appl. No.: 11/966,881

(22) Filed: Dec. 28, 2007

(51) Int. Cl. A61B 18/14

(52) U.S. Cl. 606/34

(57) ABSTRACT

Apparatus and methods for safely performing electrosurgery on a patient by evenly distributing electric current density at a return electrode unit having a plurality of concentric return electrodes. In an embodiment, each electrode may be independently coupled to a passive electrical element, and each of the passive electrical elements may have a different value of capacitance, resistance or inductance, according to the configuration of the concentric return electrodes, to provide the even distribution of electric current density between the plurality of concentric return electrodes of the return electrode unit.

### FIELD OF THE INVENTION

The present invention generally relates to apparatus and methods for performing electrosurgery.

### BACKGROUND OF THE INVENTION

Various forms of electrosurgery are now widely used for a vast range of surgical procedures. There are two basic forms or electrosurgery, namely monopolar and bipolar, according to the configuration of the electrosurgical system which determines the path of electrical energy flow vis-a-vis the patient. In the bipolar configuration, both the active electrode and the return electrode are located adjacent to a target tissue of the patient, i.e., the electrodes are in close proximity to each other, and current flows between the electrodes locally at the surgical site. In monopolar electrosurgery, the active electrode is again located at the surgical site; however, the return electrode, which is typically much larger than the active electrode, is placed in contact with the patient at a location on the patient's body that is remote from the surgical site. In monopolar electrosurgery, the return electrode is typically accommodated on a device which may be referred to as a dispersive pad, and the return electrode may also be known as the, dispersive-, patient-, neutral-, or grounding electrode.

In general, monopolar electrosurgical procedures allow a large range of tissue effects. In monopolar electrosurgery, current from an electrosurgical generator typically flows through an active electrode and into target tissue. The current then passes through the patient's body to the return electrode where it is collected and returned to the generator.

A disadvantage of monopolar electrosurgery using prior art return electrodes is the risk of burns on the patient's body at the location of the return electrode. In the case of a conventional solid return electrode, e.g., a sheet of metal foil, electric current density tends to be concentrated at the corners and/or edges of the return electrode. Concentration, or uneven distribution, of electric current density at the return electrode surface may cause excessive heating to the extent that a severe burn to the patient's tissue can result.

Some newer electrosurgical systems and applications use substantially higher current values, higher duty cycles, and/or longer delivery times for ablating, heating, or modifying target tissue, as compared with more traditional uses of electrosurgery. With these higher current densities and longer delivery times, the risk of a patient burn may be greatly increased. The present IEC 60601-2-2:2006 standard states that “No acceptable neutral electrode should exceed a 6° C. temperature rise when subjected to the required current and duration test.” The Association for the Advancement of Medical Instrumentation (“AAMI”) has published similar standards.

One approach to solving the problem of return electrode-induced patient burns has been to use multiple dispersive pads. For example, some procedures have required an increase in the number of dispersive pads from 1 to 4, or even 6, dispersive pads. However, with the increase in the number of dispersive pads, the correct placement becomes more difficult, while incorrect placement of the pads also increases the risk of a patient burn.

In an attempt to reduce edge effects and the uneven distribution of electric current density, U.S. Pat. No. 5,836,942 to Isaacson discloses a biomedical electrode having one or two conductive plates and a field of lossy dielectric material disposed between the plate(s) and the patient.

U.S. Patent Application Publication No. 20060224150 (Arts et al.) discloses a temperature regulating patient return electrode for monopolar surgery, wherein the electrode includes a positive temperature coefficient (PTC) material on the electrode surface. The PTC material responds to local temperature increases by increasing local resistance.

U.S. Patent Application Publication No. 20060074411 (Carmel et al.) discloses a dispersive electrode having conducting components that may include a central conducting plate disposed on an intermediate layer of conductive dielectric, wherein the conductive dielectric is disposed between the conducting component(s) and the patient. The central conducting plate is coupled to a generator ground, while the other conducting components, which may include components concentric with the central conducting plate, are coupled to the central conducting plate via distributed or lumped elements.

U.S. Patent Application Publication No. 20070049914 (Eggleston) discloses electrosurgical apparatus including a conductive pad having a plurality of conductive elements forming a grid and a connection device connectable to each of the plurality of the conductive elements and to an electrosurgical generator. A plurality of temperature sensors measure the temperature of a patient's skin in contact with the corresponding conductive element, and the connection device may be connected or disconnected to a conductive element when the temperature of the patient in contact with the respective conductive element reaches a predetermined level.

As can be seen, there is a need for apparatus and methods for safely performing monopolar electrosurgery using a return electrode that decreases or eliminates electrode edge effects and reduces the risk of patient burns. There is a further need for a patient return electrode for monopolar electrosurgery that decreases electrode manufacturing and disposal costs.

### SUMMARY OF THE INVENTION

According to one aspect of the invention, there is provided an electrosurgical system which includes an electrosurgical power supply and a return electrode unit configured for electrical coupling to the power supply. The return electrode unit comprises a plurality of concentric return electrodes, and the return electrode unit is configured for independently coupling each of the concentric return electrodes to the power supply.

According to another aspect of the invention, a system comprises an electrosurgical power supply, a return electrode unit configured for electrical coupling to the power supply, and a plurality of passive electrical elements electrically coupled between the return electrode unit and the power supply. The return electrode unit includes a plurality of concentric return electrodes. The system is configured for independently electrically coupling each of the passive electrical elements to a corresponding one of the concentric return electrodes. The power supply is configured for supplying electrical energy to a patient's body via an active electrode unit. The return electrode unit is configured for contacting the patient's body, for receiving the electrical energy from the patient's body, and for returning the electrical energy to the power supply via the concentric return electrodes.

According to still another aspect of the invention, there is provided an electrosurgical apparatus including a dispersive return pad having a return electrode unit, wherein the return electrode unit comprises a plurality of concentric return electrodes. The apparatus further comprises a plurality of passive electrical elements, and the apparatus is configured for independently coupling each of the concentric return electrodes to a corresponding one of the passive electrical elements.

According to yet a further aspect of the invention, a method for performing electrosurgery on a patient comprises contacting the patient's body with a return electrode unit, wherein the return electrode unit includes a plurality of concentric return electrodes; applying electrical energy to the patient's body via an active electrode unit coupled to a power supply; and receiving the electrical energy at the plurality of concentric return electrodes. Each of the plurality of concentric return electrodes is independently coupled to the power supply via a corresponding one of a plurality of passive electrical elements.

These and other features, aspects, and advantages of the present invention may be further understood with reference to the drawings, description, and claims which follow.

### BRIEF DESCRIPTION OF THE DRAWINGS

FIG. 1 is a block diagram schematically representing an electrosurgical system having concentric return electrodes and a passive element module, according to an embodiment of the invention;

FIG. 2 is a block diagram schematically representing an electrosurgical system including concentric return electrodes and an electrode monitoring unit, according to an embodiment of the invention;

FIG. 3 is a block diagram schematically representing an electrosurgical system including a return electrode unit having concentric return electrodes coupled to passive electrical elements, according to another embodiment of the invention;

FIG. 4 is a block diagram schematically representing an electrosurgical system including an electrode monitoring unit and a plurality of concentric return electrodes, according to an embodiment of the invention;

FIG. 5 schematically represents an electrosurgical system including a plurality of concentric return electrodes and a temperature monitoring unit in communication with a plurality of temperature sensors, according to another embodiment of the invention;

FIG. 6A schematically represents a return electrode unit, as seen in plan view, including a plurality of concentric return electrodes, according to an embodiment of the invention;

FIG. 6B schematically represents a dispersive return pad, as seen in plan view, including a return electrode unit, according to an embodiment of the invention;

FIG. 7A schematically represents a dispersive return pad including a return electrode unit having a bare metal surface, as seen in side view, according to an embodiment of the invention;

FIG. 7B schematically represents a dispersive return pad having a return electrode unit and an adhesive, as seen in side view, according to another embodiment of the invention;

FIG. 7C schematically represents a dispersive return pad including a return electrode unit and a cooling element, as seen in side view, according to another embodiment of the invention;

FIG. 8 schematically represents a monopolar electrosurgical procedure for treating a patient, according to an embodiment of the invention;

FIG. 9 is a flow chart schematically representing a series of steps involved in a method for performing electrosurgery, according to another embodiment of the invention; and

FIG. 10 is a flow chart schematically representing a series of steps involved in a method for preventing patient burns during an electrosurgical procedure, according to another embodiment of the invention.

### DETAILED DESCRIPTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

Broadly, the present invention provides methods and apparatus for performing monopolar electrosurgical procedures in a safe and effective manner while preventing patient burns. Patient burns are known to occur using apparatus and methods of the prior art due to uneven distribution of electric current density, resulting in hot spots, over the surface of solid prior art return electrodes. In contrast to prior art devices, return electrode units of the instant invention are configured for evenly distributing electric current density over a plurality of concentric return electrodes of the return electrode unit. Such even distribution of electric current density eliminates the formation of hot spots at the return electrode unit, thereby preventing patient burns. The present invention may also permit higher total current density at the return electrode, and, for a given procedure/electric power usage, the use of a return electrode unit having a smaller surface area as compared with conventional return electrodes. The present invention may also permit the use of fewer return pads (e.g., a single return pad) for a given procedure/electric power usage, as compared with prior art procedures using more (e.g., several) conventional return pads.

Some prior art electrosurgical return electrodes have used a field of lossy dielectric material disposed between the electrode(s) and the patient, or a positive temperature coefficient (PTC) material on the electrode surface, to prevent edge effects (which may cause patient burns). Other prior art return electrodes have electrode(s) coupled to a central conducting plate via resistive and/or capacitive elements to provide voltage distribution. Still other prior art return electrodes have used an intermediate layer of conductive dielectric, disposed between conducting elements and the patient, for voltage distribution.

Unlike electrosurgical return electrodes of the prior art, in an embodiment of the present invention there is provided an electrosurgical system including a return electrode unit and a plurality of passive electrical elements, wherein the return electrode unit comprises a plurality of concentric return electrodes, and each of the concentric return electrodes is independently coupled to a corresponding one of the passive electrical elements, such that the electric current density at the return electrode unit may be evenly distributed between the various concentric return electrodes. Advantageously, such even electric current density as provided by apparatus and methods of the instant invention decreases the risk of patient burns and increases patient safety, as compared with prior art devices.

The methods and apparatus of the instant invention may find many applications in the field of biomedical electrodes, including a broad range of monopolar electrosurgical procedures. Such procedures may involve, for example, cutting and/or coagulation during general surgery, as well as various cosmetic procedures, and the like.

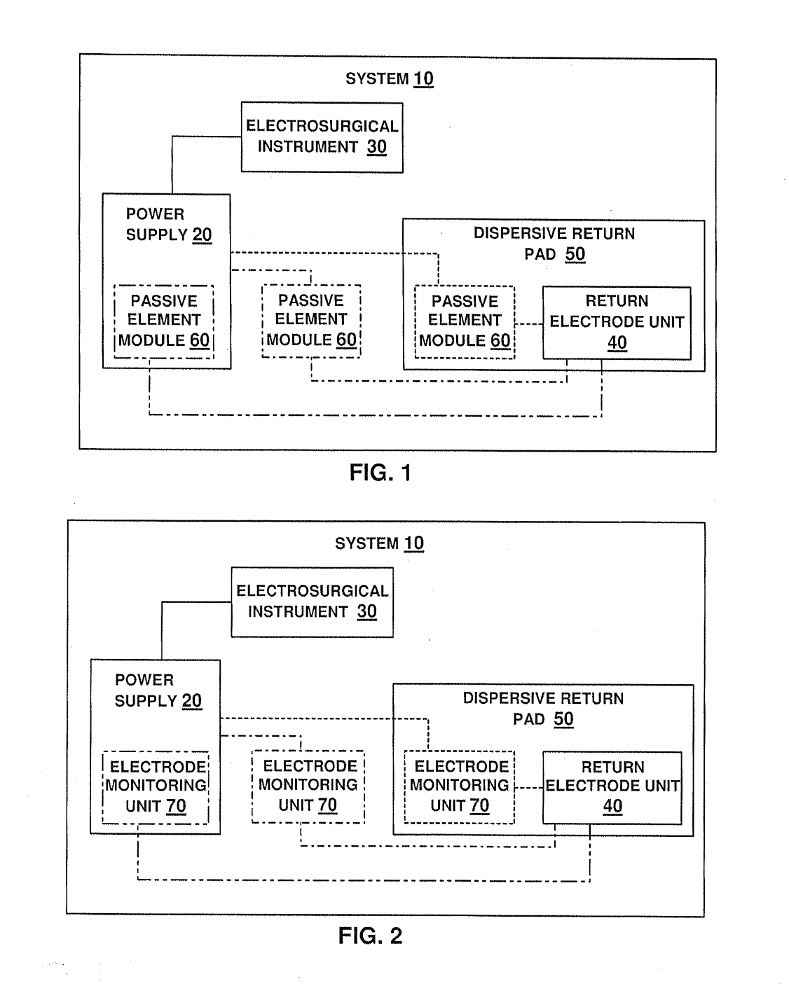

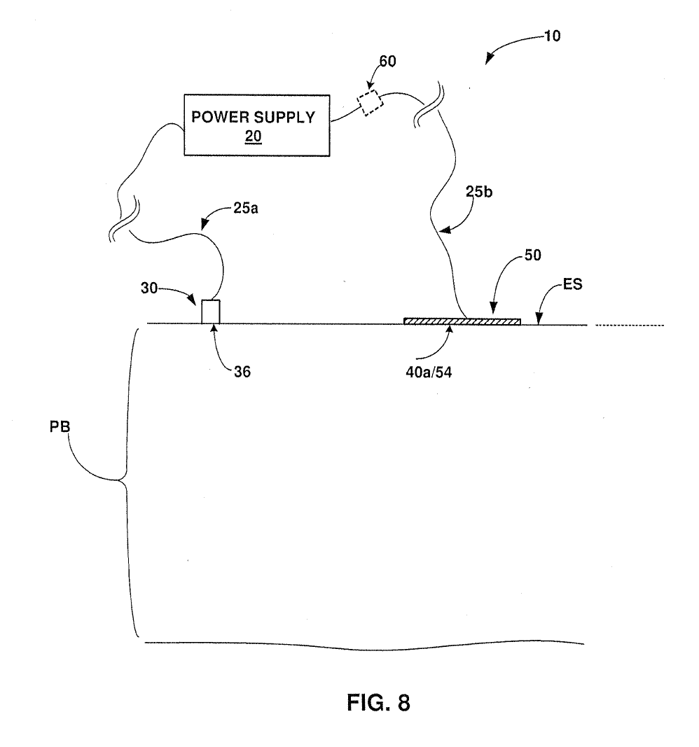

FIG. 1 is a block diagram schematically representing an electrosurgical system for treating a patient, according to an embodiment of the invention. Electrosurgical system 10 of FIG. 1 may include an electrosurgical generator or power supply 20, an electrosurgical instrument 30, and a dispersive return pad 50. Electrosurgical system 10 may be configured for monopolar electrosurgery. Power supply 20 may be configured for supplying electrical energy, such as radiofrequency (RF) alternating current, to electrosurgical instrument 30. Electrosurgical instrument 30 may be configured for electrical coupling to power supply 20, and for applying electrical energy to a patient's body or tissue(s) during a procedure. An electrosurgical procedure using an electrosurgical instrument 30 is schematically represented in FIG. 8, infra.

With further reference to FIG. 1, dispersive return pad 50 may include a return electrode unit 40. Dispersive return pad 50 may be configured for accommodating return electrode unit 40 and for contacting a patient's body. System 10 may further include a passive element module 60. System 10 may be configured for electrical coupling of return electrode unit 40 to power supply 20 via passive element module 60. In various embodiments and system architectures, passive element module 60 may be integral with power supply 20, integral with return electrode unit 40, or a separate component, as indicated by the broken lines in FIG. 1. In an embodiment, passive element module 60 may be integral with a cable 25b (see, for example, FIG. 8) for coupling return electrode unit 40 to power supply 20.

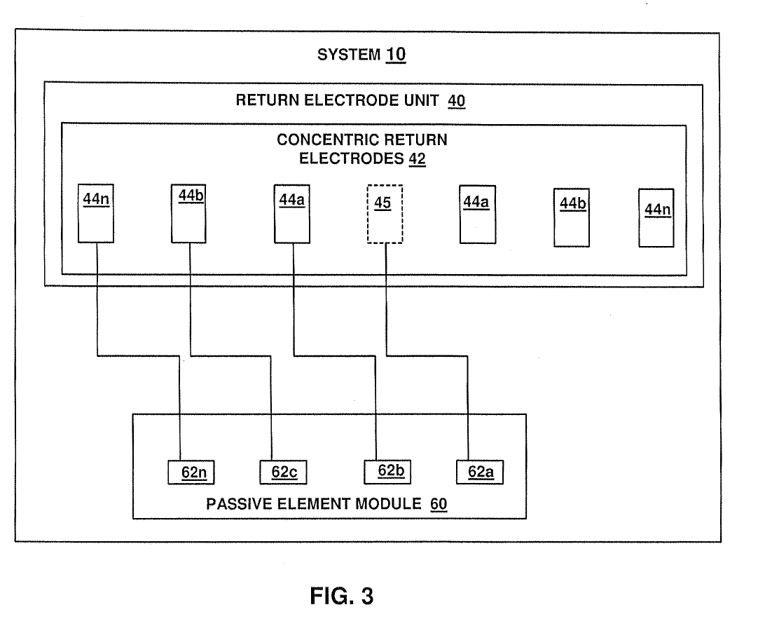

Return electrode unit 40 may be configured for contacting a patient's body with each of concentric return electrodes 42 (see, for example, FIGS. 3, 5, and 6A). Return electrode unit 40 and passive element module 60 may be configured for providing the even distribution of electric current density between each of concentric return electrodes 42 while return electrode unit 40 is receiving electrical energy from the patient's body during a procedure.

FIG. 2 is a block diagram schematically representing an electrosurgical system, according to another aspect of the invention. Electrosurgical system 10 of FIG. 2 may include an electrosurgical generator or power supply 20, an electrosurgical instrument 30, and a dispersive return pad 50, substantially as described for the embodiment of FIG. 1. Electrosurgical system 10 of FIG. 2 may further include a passive element module 60 substantially as shown in FIG. 1 (passive element module 60 is omitted from FIG. 2 for the sake of clarity). System 10 may be configured for electrically coupling return electrode unit 40 to power supply 20 via passive element module 60 (see, for example, FIGS. 1, 3, and 8).

System 10 of FIG. 2 may still further include an electrode monitoring unit 70. Electrode monitoring unit 70 may be configured for monitoring, e.g., in real time, a condition of at least one of concentric return electrodes 42 (see, for example, FIGS. 3, 4, and 5). Such an electrode condition may include, for example, temperature or current density at one or more of concentric return electrodes 42. Return electrode unit 40 may be configured for safely receiving electrical energy from a patient's body during an electrosurgical procedure, so that a burn to the patient's body may be avoided.

FIG. 3 is a block diagram schematically representing an electrosurgical system, according to another embodiment of the invention. Electrosurgical system 10 may include a return electrode unit 40 and a passive element module 60. Return electrode unit 40 may include a plurality of concentric return electrodes 42. Concentric return electrodes 42 may include a plurality of annular return electrodes 44a-n. In some embodiments, concentric return electrodes 42 may optionally further include a non-annular, center return electrode 45. In FIG. 3, return electrode unit 40 is represented in sectional view, such that annular return electrodes 44a-n appear on each side of center electrode 45. Return electrode unit 40 may be configured or adapted for directly contacting the body, e.g., skin or other tissue, of a patient during an electrosurgical procedure.

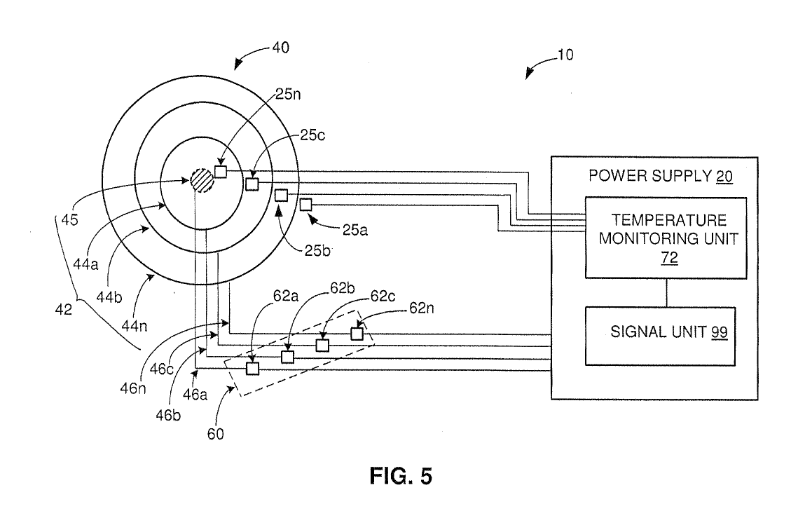

With further reference to FIG. 3, return electrode unit 40 may be configured for independently coupling each of concentric return electrodes 42 to power supply 20 (see, for example, FIGS. 1-2, and 5). Passive element module 60 may include a plurality of passive electrical elements 62a-n. Each of annular return electrodes 44a-n, and (when present) center return electrode 45, may be independently electrically coupled directly to a corresponding one of passive electrical elements 62a-n (see, for example, FIG. 5). In an embodiment, passive electrical elements 62a-n may be coupled between return electrode unit 40 and power supply 20. Each of passive electrical elements 62a-n may be independently coupled to power supply 20 (see, for example FIG. 5). Return electrode unit 40 may be coupled to power supply 20 via a cable 25b (see, for example, FIG. 8). In an embodiment, passive electrical elements 62a-n may be integral with cable 25b, e.g., as components of a connection block (not shown), the latter being well known in the art. In another embodiment, passive element module 60 may be integral with power supply 20 (see, for example, FIG. 1).

Each of passive electrical elements 62a-n may comprise, for example, a capacitor, a resistor, an inductor, or a combination thereof. Each of passive electrical elements 62a-n may have a different value of capacitance, inductance, or resistance. Each of concentric return electrodes 42 may be configured for receiving electrical energy from the patient's body during a monopolar electrosurgical procedure, and for returning the electrical energy to power supply 20. The configuration of return electrode unit 40 having each of concentrically arranged annular return electrodes 44a-n, and (optionally) axially disposed center return electrode 45, independently (e.g., separately) coupled to a corresponding one of passive electrical elements 62a-n, may promote the even distribution of electric current density between concentric return electrodes 42, giving uniform current density across the electrode unit 40.

A value of capacitance, inductance, or resistance of each of passive electrical elements 62a-n may be pre-set, for example, according to the configuration of return electrode unit 40 (e.g., the number, diameter, and composition of the various concentric return electrodes 42), such that electric current density may be evenly distributed at each of concentric return electrodes 42. By evenly distributing electric current density at each of concentric return electrodes 42, the instant invention allows procedures to be performed with a smaller dispersive return pad for a given procedure, while eliminating localized high return electrode current densities (e.g., edge effects) and preventing patient burns.

With still further reference to FIG. 3, the size and geometry of return electrode unit 40, and the number and configuration of annular return electrodes 44a-n may vary, for example according to: the composition of annular return electrodes 44a-n; the presence or absence, composition, and geometry of center return electrode 45; the values of passive electrical elements 62a-n; the weight and age of the patient; the nature of the electrosurgical procedure, etc. Typically, return electrode unit 40 may include up to about twenty-five (25) or more annular electrodes 44a-n, or from about five (5) to about eighteen (18) annular electrodes 44a-n, or from about five (5) to about fifteen (15) annular electrodes 44a-n.

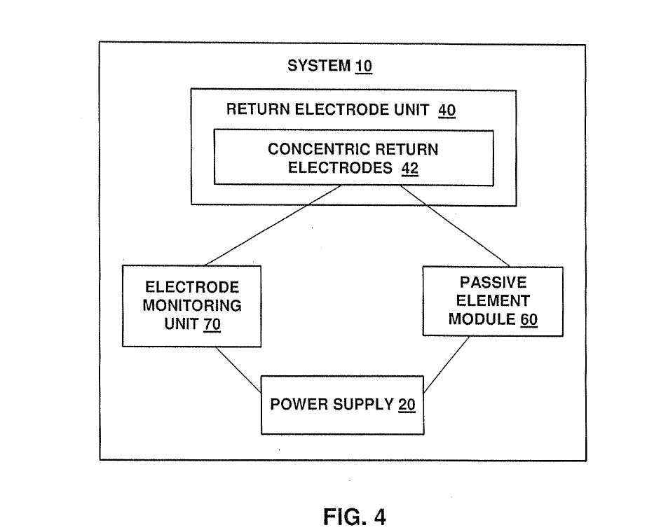

FIG. 4 is a block diagram schematically representing an electrosurgical system, according to another embodiment of the invention. Electrosurgical system 10 of FIG. 4 may include a return electrode unit 40, a passive element module 60, an electrode monitoring unit 70, and a power supply 20. Return electrode unit 40 may be coupled to power supply 20 via passive element module 60. Return electrode unit 40 may include a plurality of concentric return electrodes 42, substantially as described for other embodiments (see, for example, FIGS. 2, 5 and 6A). Although FIG. 4 shows only a single line connecting return electrode unit 40 to passive element module 60, each of concentric return electrodes 42 may be separately, or independently, coupled to a passive electrical element 62a-n (see, for example, FIGS. 2 and 5).

With further reference to FIG. 4, return electrode monitoring unit 70 may be in electrical communication with at least one of concentric return electrodes 42. In an embodiment, each of concentric return electrodes 42 may be in electrical communication with return electrode monitoring unit 70. Return electrode monitoring unit 70 may be configured for monitoring at least one electrode condition of concentric return electrodes 42. As non-limiting examples, return electrode monitoring unit 70 may be configured for monitoring, in real time, an electrode condition, such as electrode temperature, of each of concentric return electrodes 42.

With still further reference to FIG. 4, electrode monitoring unit 70 may be in electrical communication with power supply 20, and power supply 20 may be shut off or adjusted if a maximum threshold value of a monitored electrode condition is exceeded.

FIG. 5 schematically represents an electrosurgical system, according to another embodiment of the invention. Electrosurgical system 10 of FIG. 5 may include a return electrode unit 40, first through n^{th} connectors 46a-n, a passive element module 60, and a power supply 20. Return electrode unit 40 may include concentric return electrodes 42, which may include a plurality of annular return electrodes 44a-n. Concentric return electrodes 42 may further include, in some embodiments, a center return electrode 45. Return electrode unit 40 may still further include various other elements and features, for example, as described hereinabove.

With further reference to FIG. 5, return electrode unit 40 may be configured for contacting a patient's body and for receiving electrical energy from the patient's body for the return of the electrical energy to power supply 20. As shown in FIG. 5, passive element module 60 may be electrically coupled between return electrode unit 40 and power supply 20. Passive element module 60 may include a plurality of passive electrical elements 62a-n. Each of annular return electrodes 44a-n, and center return electrode 45 (if present), may be independently coupled to a corresponding one of passive electrical elements 62a-n via a separate, corresponding one of first through nth connectors 46a-n. In an embodiment, first through nth connectors 46a-n may comprise, for example, a wire; and first through nth connectors 46a-n may be housed within, or integral with, a cable 25b (see, for example FIG. 8). Each of passive electrical elements 62a-n may be independently coupled to power supply 20. Each of passive electrical elements 62a-n may include at least one capacitor, at least one resistor, at least one wire, at least one inductor, or a combination thereof. Each of passive electrical elements 62a-n may have a different value of capacitance, resistance, or inductance, such that electric current density may be evenly distributed between concentric return electrodes 42.

System 10 may further include a plurality of temperature sensors 25a-n configured for providing temperature data for concentric return electrodes 42. Temperature sensors 25a-n may each comprise, for example, a thermocouple, a resistance temperature detector (RTD), or a thermistor. Temperature sensors 25a-n may be in electrical communication with temperature monitoring unit 72, wherein temperature monitoring unit 72 receives temperature data, e.g., a temperature value of each of concentric return electrodes 42, from temperature sensors 25a-n. As shown, temperature monitoring unit 72 may be integral with power supply 20.

With still further reference to FIG. 5, system 10 may be configured to shut off or adjust power supply 20 if a maximum threshold temperature for one or more concentric return electrodes 42 is exceeded. In another embodiment, system 10 may be configured to shut off or adjust power supply 20 in response to a mismatch in electrode temperature values between concentric return electrodes 42.

System 10 may further include a signal unit 99 for signaling an operator or other medical personnel. As shown, signal unit 99 may also be integral with power supply 20; however, other locations for signal unit 99 are also within the scope of the invention. Signal unit 99 may be in electrical communication with temperature monitoring unit 72, and signal unit 99 may be configured for providing a visual or audible signal, e.g., in response to a mismatch in electrode temperature values between concentric return electrodes 42, or if a maximum threshold temperature for one or more concentric return electrodes 42 is exceeded.

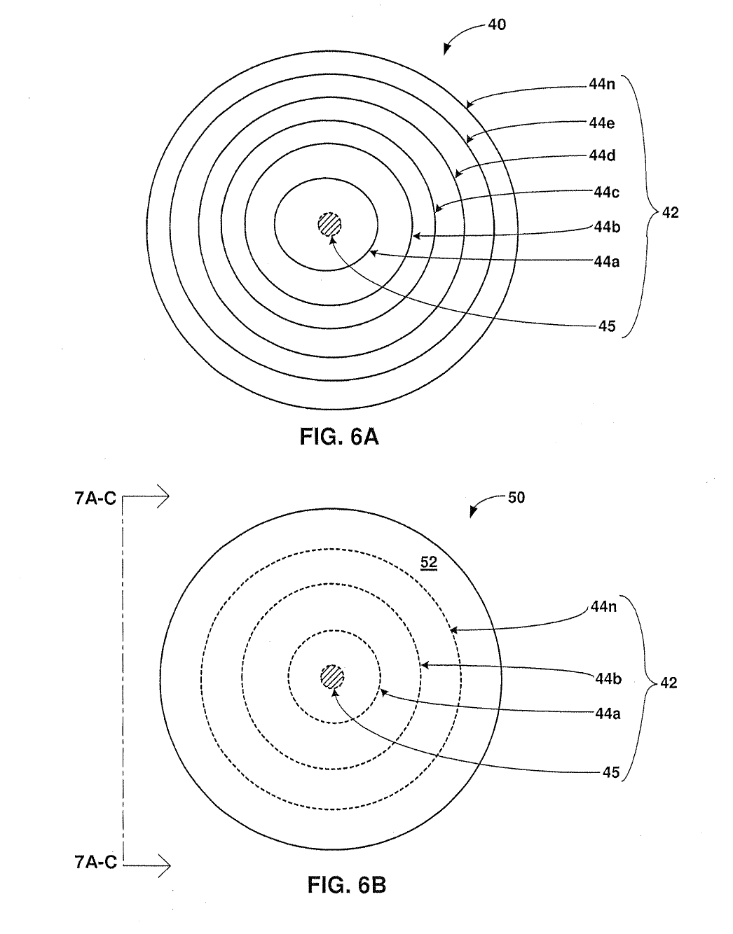

FIG. 6A schematically represents a return electrode unit in plan view, according to an embodiment of the invention. Return electrode unit 40 may include a plurality of concentric return electrodes 42. More specifically, return electrode unit 40 may include first through n^{th} annular return electrodes 44a-n. Typically, each of first through n^{th} annular return electrodes 44a-n may be in the form of an entire or unbroken ring. As shown, each of first through n^{th} annular return electrodes 44a-n may be at least substantially circular. In an embodiment, return electrode unit 40 may typically include from about five (5) to about twenty-five (25) annular return electrodes 44a-n, usually from about five (5) to eighteen (18) annular return electrodes 44a-n, and in some embodiments from about five (5) to fifteen (15) annular return electrodes 44a-n. However, it is to be understood that other numbers of annular return electrodes 44a-n are also within the scope of the invention. Return electrode unit 40 may be at least substantially planar before and/or during use thereof. Return electrode unit 40 may be flexible and deformable so as to allow contact between each of concentric return electrodes 42 and a planar or non-planar surface of a patient's body.

With further reference to FIG. 6A, each of first through n^{th} annular return electrodes 44a-n may comprise an electrically conductive metal, such as stainless steel, gold, silver, copper, aluminum, zinc, lead, tin, iron, carbon or any alloys using these elements; and each of annular return electrodes 44a-n may have a bare metal surface for contacting a patient's body. In an embodiment, a metal surface of annular return electrodes 44a-n may be at least partially covered by an adhesive.

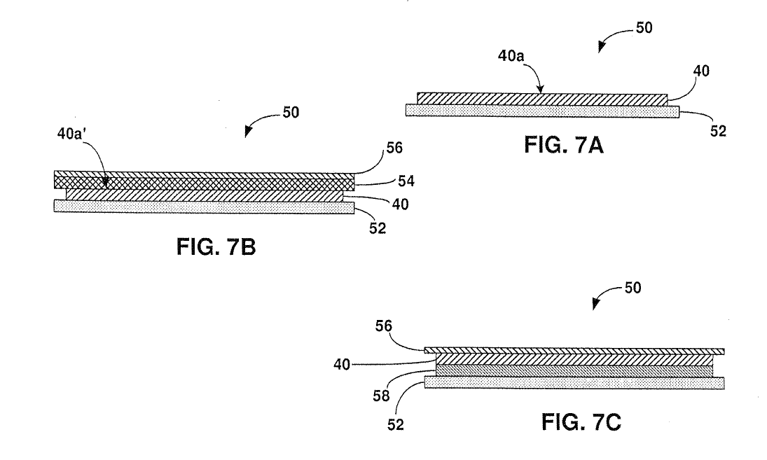

Return electrode unit 40 may be incorporated in a dispersive return pad 50 having a support layer 52 (see, for example, FIGS. 6B and 7A-C). In some embodiments, concentric return electrodes 42 may optionally further include a non-annular, center return electrode 45. Center return electrode 45 may be at least substantially circular in outline. Each of concentric return electrodes 42, including center return electrode 45 (if present), may comprise an electrically conductive metal. As non-limiting examples, each of annular return electrodes 44a-n may comprise a ring of metal foil, a ring of flattened metal wire, or a ring of metal ribbon.

FIG. 6B schematically represents a dispersive return pad in plan view, according to another aspect of the invention. Dispersive return pad 50 may include a support layer 52 and a return electrode unit 40. In use, return electrode unit 40 may be oriented towards the patient and in contact with the patient's body, while support layer 52 may support or cover return electrode unit 40. Return electrode unit 40 may include annular return electrodes 44a-n, and, in some embodiments, a non-annular, center return electrode 45.

In use, annular return electrodes 44a-n and center return electrode 45 (if present) may typically be at least partially obscured by support layer 52. Support layer 52 may comprise an electrically non-conductive material (such as, for example, Teflon, Polyamide, FR4, G10, Nylon, Polyester, Kapton, Silicone, rubber), and may serve to electrically insulate concentric return electrodes 42 from medical personnel, the patient, medical instruments, equipment, and the like. Dispersive return pad 50 may further include a protective layer 56 (see, for example, FIGS. 7A-C). In an embodiment, dispersive return pad 50 may still further include an adhesive 54 (see, for example, FIGS. 7B-C). Return electrode unit 40 of FIG. 6B may have various characteristics, elements, and features as described herein, for example, with respect to FIGS. 2, 5, and 6A.

The size or area of dispersive return pad 50 may be adapted or varied according to factors such as the nature of the electrosurgical procedure, patient characteristics, as well as the power and duty cycle of electrosurgical apparatus used to apply the electrical energy to the patient, etc.

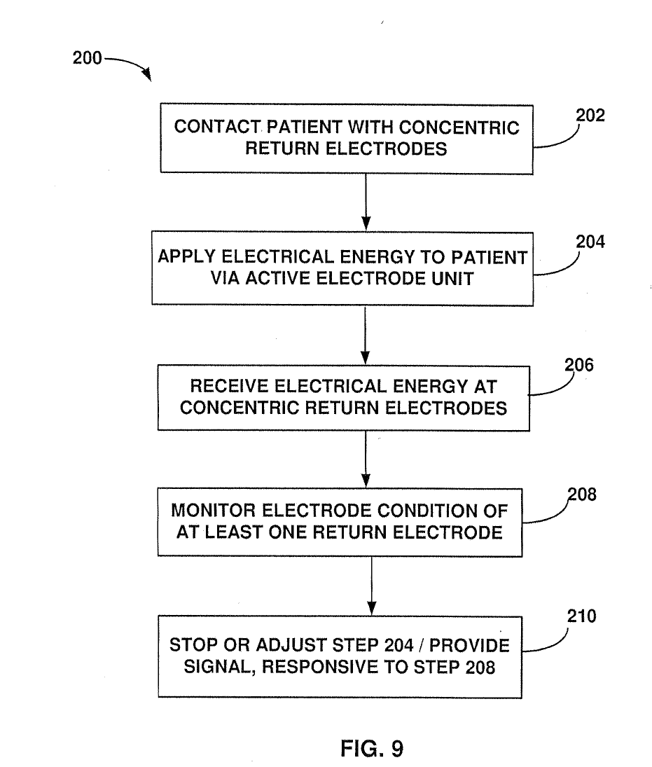

In an embodiment, dispersive return pad 50 and return electrode unit 40 may be compatible with, and used in conjunction with, a contact monitoring unit (see, for example, FIGS. 9A-B, 10A-B, 11A, and 11C) for monitoring contact between the patient's body and dispersive return pad 50 or return electrode unit 40.

FIG. 7A schematically represents a dispersive return pad, as seen in side view along the lines 7A-C-7A-C of FIG. 6B, according to an embodiment of the invention. Dispersive return pad 50 of FIG. 7A may include a support layer 52, and a return electrode unit 40 disposed on, or adjacent to, support layer 52. Support layer 52 may comprise an electrically non-conductive or electrically insulating material. As shown in FIG. 7A, return electrode unit 40 may include a patient-contacting surface 40a, wherein patient-contacting surface 40a may comprise a bare metal surface of concentric return electrodes 42, and such a bare metal patient-contacting surface 40a may be configured for directly contacting the patient's body during a procedure. That is to say, in the embodiment of FIG. 7A, all or part of patient-contacting surface 40a of return electrode unit 40 may be devoid of adhesive, gel, or any other material; and dispersive return pad 50 may be configured for bare metal contact of return electrode unit 40 on the patient's body (for example, skin or other tissue).

Return electrode unit 40 may include a plurality of concentric return electrodes 42 (see, for example, FIG. 6A), and return electrode unit 40 may have other characteristics, features and elements as described herein, for example, with reference to FIGS. 2 and 6A. As a non-limiting example, return electrode unit 40 may comprise up to about 25 or more concentrically arranged return electrodes 42. Dispersive return pad 50 may further include a protective layer 56 (see, for example, FIGS. 7B-C), which may protect return electrode unit 40 or other components of dispersive return pad 50 during transportation or storage thereof (protective layer 56 is not shown in FIG. 7A).

FIG. 7B schematically represents a dispersive return pad, as seen in side view along the lines 7A-C-7A-C of FIG. 6B, according to another embodiment of the invention. Dispersive return pad 50 of FIG. 7B may include a support layer 52; a return electrode unit 40 disposed on, or adjacent to, support layer 52; and an adhesive 54 disposed on, or adjacent to, return electrode unit 40. Adhesive 54 may comprise an electrically conductive material. In an embodiment, adhesive 54 may be in contact with a patient contacting side 40a′ of return electrode unit 40. Adhesive 54 may comprise, for example, a polyacrylate- or polyolefin-based pressure-sensitive adhesive, or a hydrogel adhesive.

In an embodiment, adhesive 54 may be specifically selected so as to have a low or very low electrical resistivity. For example, adhesive 54 may be selected to have a specific resistivity value of <0.1 Ohm.m, typically a specific resistivity value of 0.01 Ohm.m or less, usually a specific resistivity value of 0.001 Ohm.m or less, and preferably a specific resistivity value of 0.0001 Ohm.m or less. In an embodiment, adhesive 54 may have a specific resistivity value in the range of from about 0.00001 to 0.00000001 Ohm.m or less.

In an embodiment, adhesive 54 may be aligned or flush with the perimeter of dispersive pad 50. In an embodiment, adhesive 54 may extend over the entire surface of dispersive pad 50. In an embodiment, adhesive 54 may comprise a strip or band (not shown) of adhesive material, which may be disposed at or near a periphery of dispersive return pad 50. Such a strip or band of adhesive material may be disposed radially outward from return electrode unit 40, such that adhesive 54 does not contact return electrode unit 40. Adhesive 54 may be an amorphous material.

Dispersive return pad 50 may further include a protective layer 56, which may be disposed on adhesive 54. Protective layer 56 may protect components of dispersive return pad 50 prior to use of dispersive return pad 50. Protective layer 56 may be configured for facile removal thereof prior to use of dispersive return pad 50.

FIG. 7C schematically represents a dispersive return pad, as seen in side view along the lines 7A-C-7A-C of FIG. 6B, according to another embodiment of the invention. As shown, dispersive return pad 50 of FIG. 7C may include a support layer 52, a return electrode unit 40, and a cooling element 58. Cooling element 58 may be disposed on, or adjacent to, return electrode unit 40. As shown, cooling element 58 may be disposed between return electrode unit 40 and support layer 52. A cooling mechanism for cooling element 58 may comprise an active cooling mechanism or a passive cooling mechanism. As non-limiting examples, cooling element 58 may comprise: a forced-air cooling mechanism, liquid cooling, a chemical (endothermic) cooling mechanism, a heat exchanger, a thermoelectric cooler, or a pocket for accommodating a cooling pack (none of which are shown). Dispersive return pad 50 of FIG. 7C may further include an adhesive 54, substantially as described herein with reference to FIG. 7B.

Dispersive return pads 50 of the invention, such as those of FIGS. 7A-C, may be configured for independently coupling each of concentric return electrodes 42 to a corresponding one of passive electrical element 62a-n (see, for example, FIGS. 2 and 5) to provide even distribution of electric current density between concentric return electrodes 42 of return electrode unit 40 during use of dispersive return pad 50. FIGS. 7A-C may not be drawn to scale. Architectures other than those shown in FIGS. 7A-C for dispersive return pads 50 are also within the scope of the invention.

FIG. 8 schematically represents a monopolar electrosurgical procedure for treating a patient, according to an embodiment of the invention. Such a procedure may involve placing a dispersive return pad 50 in contact with the patient's body, PB. As shown, dispersive return pad 50 may be configured for contacting an external surface, ES, of the patient's body, for example, skin. Dispersive return pad 50 may be conformable to a non-planar external surface of various parts of the patient's body. Dispersive return pad 50 may include a return electrode unit 40 having a plurality of concentric return electrodes 42, as well as other elements and features as described herein (for example, with reference to FIGS. 6B and 7A-C).

In an embodiment, dispersive return pad 50 may have a bare metal patient-contacting surface 40a (see, for example, FIG. 7A) comprising a surface of each of concentric return electrodes 42 of return electrode unit 40, and the bare metal patient-contacting surface 40a may be placed in contact with the patient's body. In another embodiment, dispersive return pad 50 may be placed in contact with the patient's body via an adhesive 54 (see, for example, FIG. 7B). An electrosurgical instrument 30 and dispersive return pad 50 may be coupled to opposite poles of power supply 20, via cables 25a and 25b, respectively. In an embodiment, passive element module 60 (see, for example, FIG. 3) may be integral with cable 25b. Electrosurgical instrument 30 may comprise or may be a component of an electrosurgical handpiece, as is well known in the art.

Power supply 20 may be configured for supplying electrical energy, for example, high frequency (e.g., RF) alternating current, to the patient's body. During the procedure electrical energy may be applied to the patient's body via electrosurgical instrument 30, and the electrical energy may be received by return electrode unit 40 of dispersive return pad 50. Electrosurgical instrument 30 may include a treatment face 36, and treatment face 36 may be configured for contacting the patient's body during a procedure.

With further reference to FIG. 8, electrosurgical instrument 30 may be configured for performing various procedures on the patient, which may involve, for example, heating, liquefaction, ablation, cutting, coagulation, or fulguration, etc. of a target tissue of the patient. In a non-limiting example, electrosurgical instrument 30 may be configured for treating the skin of the patient, and the procedure may involve modification of the texture, coloration, hirsuteness, smoothness, etc. of the patient's skin. In another non-limiting example, electrosurgical instrument 30 may be configured for selectively heating a target tissue of the patient, such as subcutaneous fat, and the procedure may involve non-invasive lipolysis beneath the patient's skin.

FIG. 9 is a flow chart schematically representing steps in a method for performing electrosurgery on a patient, according to another embodiment of the invention. Step 202 of method 200 may involve contacting a patient's body with a plurality of concentric return electrodes. Typically, step 202 may involve contacting an external surface, such as the skin surface, of the patient's body with the concentric return electrodes. The concentric return electrodes may be housed within or on a dispersive return pad substantially as described herein, for example, with reference to FIGS. 6B and 7A-C (supra). The dispersive return pad may be configured for contacting such an external surface of the patient's body. In an embodiment, the dispersive pad may include an adhesive, such that an adhesive material may be disposed on the return electrode unit. In an embodiment, step 202 may involve contacting the patient's body with such an adhesive material applied to at least a portion of the return electrode unit. In another embodiment, step 202 may involve contacting the patient's body with a bare metal surface of at least a portion of the return electrode unit. Such a bare metal surface may be a patient-contacting surface comprising a surface of each of the plurality of concentric return electrodes of the return electrode unit.

Step 204 of method 200 may involve applying electrical energy to the patient via an active electrode unit. The active electrode unit may be a component of an electrosurgical instrument (see, for example, FIGS. 1, 2 and 8). During step 204, electrical energy may be applied to a target tissue, e.g., skin, adipose tissue, connective tissue, cardiovascular tissue, joint tissue, gastrointestinal tissue, endocrine tissue, nervous tissue, etc., to effect treatment of the patient.

Step 206 may involve receiving the electrical energy, from the patient's body, via the concentric return electrodes. Each of the concentric return electrodes may be independently coupled to a passive electrical element to promote the even distribution of electric current density between each of the concentric return electrodes, wherein each of the passive electrical elements may have a different value of capacitance, resistance, or inductance. Each of the passive electrical elements may be independently coupled to the power supply.

In an embodiment, optional step 208 may involve monitoring an electrode condition of at least one of the concentric return electrodes. As a non-limiting example, an electrode condition comprising temperature may be monitored by at least one temperature sensor coupled to an electrode temperature monitoring unit (see, for example, FIG. 5).

In embodiments involving step 208, step 210 may involve stopping or adjusting step 204 if an electrode condition exceeds a maximum threshold value. Step 210 may involve an automatic shut down or adjustment of the power supply. Alternatively, or additionally, step 210 may involve signaling an operator or other medical personnel, via a signal unit (see, for example, FIG. 5), that the maximum threshold value has been exceeded. In an embodiment, the maximum threshold value may be set such that there is a lag between the maximum threshold value being exceeded and a condition that may actually harm a patient.

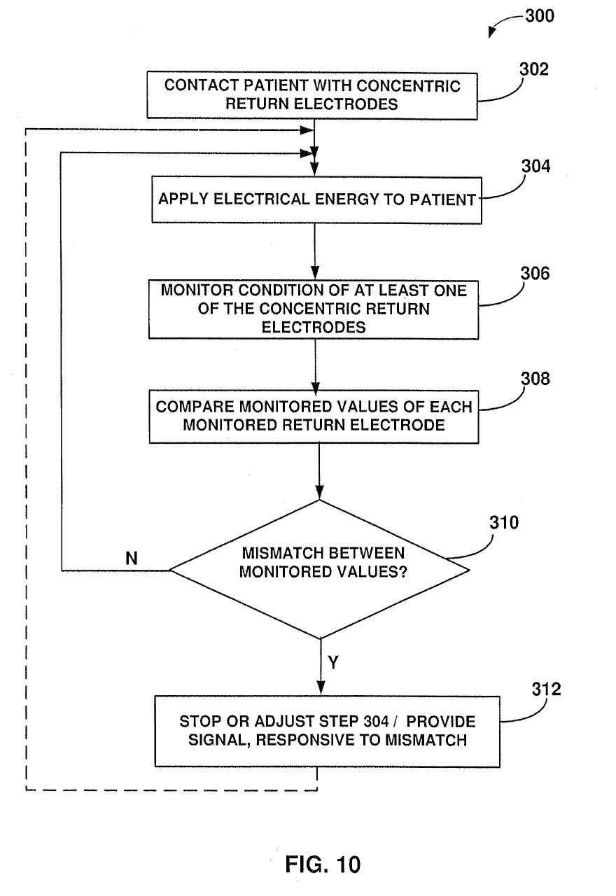

FIG. 10 is a flow chart schematically representing steps in a method for performing electrosurgery, according to another embodiment of the invention. Step 302 of method 300 may involve contacting a patient with a plurality of concentric return electrodes, substantially as described for step 202 of method 200 (supra).

Step 304 may involve applying electrical energy to the patient. Step 306 may involve monitoring an electrode condition of at least one of the concentric return electrodes, substantially as described for step 208 of method 200 (supra). In an embodiment, an electrode condition of each of the concentric return electrodes may be monitored during step 306.

Step 308 may involve comparing values of a monitored electrode condition for each of the concentric return electrodes. As a non-limiting example, temperature values for each return electrode may be compared with a reference temperature value. In an embodiment, the reference temperature value may be a monitored temperature of a reference electrode, for example, the temperature of the reference electrode may be repeatedly monitored during a procedure for comparison with the temperature values of other return electrodes. As a non-limiting example, the reference electrode may be the radially innermost of the concentric return electrodes. In various embodiments, the radially innermost concentric return electrode may be an annular return electrode, or a non-annular center return electrode.

In another embodiment, the reference temperature value may be “factory” pre-set; for example, the reference temperature value may be fixed during manufacture or assembly of a power supply having a temperature monitoring unit (see, for example, FIG. 5). In another embodiment, the reference temperature value may be set “on the fly” by medical personnel prior to or during an electrosurgical procedure. For example, the reference temperature value may be set (e.g., via controls (not shown) on the power supply) by medical personnel according to factors such as the characteristics (e.g., weight, age, BMI) of the patient, the nature of the procedure to be performed, and the like.

At decision block or step 310, if a mismatch exists between two or more of the concentric return electrodes (Y) for the monitored temperature or condition, step 304 may be stopped or adjusted, whereby the application of electrical energy to the patient, and concomitantly, receipt of electrical energy at the return electrode unit, may immediately cease or be decreased. Thereafter, flow may proceed back to block or step 304. Conversely, if there is no mismatch (N) between the compared information, step 312 may be omitted and flow may proceed back to block 304 for reiteration.

In an embodiment, medical personnel may control, e.g., within a defined range of stringencies, the level of stringency with which a mismatch of electrode condition between the various concentric return electrodes is to be determined at block 310. For example, if the power supply is configured to register a mismatch (e.g., a temperature difference) when a discrepancy of ±x % is observed between a monitored return electrode and the reference return electrode, in an embodiment, a value of x may be controlled by medical personnel within a range of values for x. Such control of the value of x may be exercised, for example, according to the characteristics (e.g., dimensions, electrode configuration, and the like) of the return electrode unit, the characteristics of the electrosurgical power supply, the nature of the procedure, and the like. In other embodiments, the level of stringency with which a mismatch between values for concentric return electrodes is to be found may be pre-set at a fixed level.

The disclosed systems may be provided with instructions for use instructing the user to use the system in accordance with the disclosed methods.

It should be understood, that the foregoing relates to exemplary embodiments of the invention, none of the examples presented herein are to be construed as limiting the present invention in any way, and that modifications may be made without departing from the spirit and scope of the invention as set forth in the following claims.

What is claimed is:

1. An electrosurgical system, comprising:
an electrosurgical power supply; and
a return electrode unit configured for electrical coupling to said power supply, wherein:
said return electrode unit comprises a plurality of concentric return electrodes,
said return electrode unit is configured for independently coupling each of said plurality of concentric return electrodes to said power supply
a plurality of passive electrical elements, each of said passive electrical elements is independently coupled to said power supply; and
wherein said system is configured for independently coupling each of said plurality of concentric return electrodes to a corresponding one of said plurality of passive electrical elements.

2. The system of claim 1, wherein each of said plurality of passive electrical elements has a different value of capacitance, inductance, or resistance.

3. The system of claim 1, wherein:
said return electrode unit is configured for contacting a patient's body,
said return electrode unit is further configured for receiving electrical energy from the patient's body, and
said plurality of passive electrical elements is configured for evenly distributing electric current density between said plurality of concentric return electrodes.

4. The system of claim 1, wherein said plurality of concentric return electrodes includes from about five (5) to about twenty-five (25) annular return electrodes.

5. The system of claim 1, further comprising:
a temperature monitoring unit configured for monitoring a temperature of at least one of said concentric return electrodes, and
at least one temperature sensor in communication with said at least one concentric return electrode, wherein:
said at least one temperature sensor is in further communication with said temperature monitoring unit.

6. A system, comprising:
an electrosurgical power supply;
a return electrode unit configured for electrical coupling to said power supply; and
a plurality of passive electrical elements electrically coupled between said return electrode unit and said power supply, wherein:
said return electrode unit includes a plurality of concentric return electrodes,
said power supply is configured for supplying electrical energy to a patient's body via an active electrode unit,
said return electrode unit is configured for contacting the patient's body, for receiving said electrical energy from the patient's body, and for returning said electrical energy to said power supply via said plurality of concentric return electrodes, and
said system is configured for independently electrically coupling each of said plurality of passive electrical elements to a corresponding one of said plurality of concentric return electrodes.

7. The system of claim 6, wherein:
each of said plurality of passive electrical elements has a different value of capacitance, inductance, or resistance, and
said plurality of passive electrical elements are configured for providing an even distribution of electric current density between said plurality of concentric return electrodes.

8. The system of claim 7, further comprising:
at least one temperature sensor in communication with said return electrode unit, and
a signal unit configured for providing a signal based on a temperature value of at least one of said concentric return electrodes, wherein said temperature value is monitored via said at least one temperature sensor.

9. The system of claim 6, further comprising a cable configured for coupling said return electrode unit to said power supply, and wherein said plurality of passive electrical elements are integral with said cable.

10. Electrosurgical apparatus, comprising:
a dispersive return pad,
said dispersive return pad includes a return electrode unit,
said return electrode unit includes a plurality of concentric return electrodes, and
said apparatus further comprises a plurality of passive electrical elements, wherein said apparatus is configured for independently coupling each of said plurality of concentric return electrodes to a corresponding one of said plurality of passive electrical elements.

11. The apparatus of claim 10, wherein:
said plurality of concentric return electrodes includes a plurality of annular return electrodes,
said plurality of annular return electrodes comprises from about five (5) to about twenty-five (25) of said annular return electrodes, and
each of said annular return electrodes has an entire or unbroken circular configuration.

12. The apparatus of claim 10, wherein:
said apparatus is configured for independently coupling each of said plurality of concentric return electrodes to an electrosurgical power supply via said plurality of passive electrical elements.

13. The apparatus of claim 12, wherein:
said plurality of concentric return electrodes comprises a plurality of annular return electrodes and a non-annular, center return electrode, and
said center return electrode is axially disposed with respect to said plurality of annular return electrodes.

14. The apparatus of claim 13, wherein said return electrode unit includes a bare metal patient-contacting surface configured for directly contacting a patient's body.

15. The apparatus of claim 10, wherein:
said return electrode unit is configured for contacting a patient's body,
each of said plurality of concentric return electrodes is configured for receiving electrical energy from the patient's body,
each of said passive electrical elements has a different value of capacitance, resistance, or inductance, and
said plurality of passive electrical elements is configured for evenly distributing electric current density between said plurality of concentric return electrodes.

16. The apparatus of claim 10, further comprising:
a cooling element configured for cooling said return electrode unit, wherein:
said cooling element is disposed adjacent to said return electrode unit.

17. The apparatus of claim 10, further comprising an adhesive disposed on said return electrode unit, wherein said adhesive has a specific resistivity value less than 0.1 Ohm.m.

18. A method for performing electrosurgery on a patient, comprising:
a) contacting the patient's body with a return electrode unit, said return electrode unit including a plurality of concentric return electrodes;
b) applying electrical energy to the patient's body via an active electrode unit coupled to a power supply; and
c) receiving said electrical energy at said plurality of concentric return electrodes, wherein each of said plurality of concentric return electrodes is independently coupled to said power supply via a corresponding one of a plurality of passive electrical elements.

19. The method of claim 18, wherein each of said plurality of passive electrical elements has a different value of capacitance, resistance, or inductance, such that said plurality of passive electrical elements is configured for evenly distributing electric current density between said plurality of concentric return electrodes.

20. The method of claim 18, further comprising:
d) monitoring an electrode condition for at least one of said concentric return electrodes, and
e) responsive to said step d), stopping or adjusting said step b) if a monitored value for said electrode condition exceeds a maximum threshold value.

21. The method of claim 20, wherein said monitored value comprises electrode temperature.

22. The method of claim 20, wherein said step d) comprises: comparing said monitored value for each of said plurality of concentric return electrodes.

23. The method of claim 18, wherein:
said step b) comprises applying said electrical energy to a target tissue of the patient's body, and
the target tissue comprises skin of the patient.

24. The method of claim 18, wherein:
said step b) comprises applying said electrical energy to a target tissue of the patient's body, and
the target tissue comprises subcutaneous fat of the patient.
